(19) United States
(12) Patent Application Publication
(10) Pub. No.: US 2003/0129605 A1
(43) Pub. Date: Jul. 10, 2003

(54) Immunostimulatory activity of CpG oligonucleotides containing non-ionic methylphosophonate linkages

(76) Inventors: Dong Yu, Westboro, MA (US); Ekambar B. Kandimalla, Southboro, MA (US); Qiuyan Zhao, Southboro, MA (US); Sudhir Agrawal, Shrewsbury, MA (US)

Correspondence Address: KEOWN & ASSOCIATES, 500 WEST CUMMINGS PARK, SUITE 1200, WOBURN, MA, 01801, US

(21) Appl. No.: 10/137,687

(22) Filed: Aug. 12, 2002

Related U.S. Application Data
(60) Provisional application No. 60/288,917, filed on May 4, 2001.

(51) Int. Cl. A61K 48/00; C12Q 1/68

(52) U.S. Cl. 435/6; 514/44

(57) ABSTRACT

Bacterial DNA and synthetic oligodeoxynucleotides containing unmethylated CpG-motifs in a particular sequence context activate vertebrate immune cells. We examined the significance of negatively charged internucleoside linkages in the flanking sequences 5′ and 3′ to the CpG-motif on immunostimulatory activity. Cell proliferation and secretion of IL-12 and IL-6 in mouse spleen cell cultures, and spleen weights of mice increased significantly when a non-ionic linkage was placed at least four or more internucleoside linkages away from the CpG-motif in the 5′-flanking sequence. When the non-ionic linkage was placed closer than three internucleoside linkages in the 5′-flanking sequence to the CpG-motif, immunostimulatory activity was suppressed compared with that observed with the unmodified parent oligo. In general, the placement of non-ionic linkage in the 3′-flanking sequence to the CpG-motif either did not affect or slightly increased immunostimulatory activity compared with the parent oligo. These results have significance in understanding CpG oligonucleotide-receptor interactions and the development of potent immunomodulatory agents.

### BACKGROUND OF THE INVENTION

1. Field of the Invention

The invention relates to oligonucleotide-mediated immune stimulation. More particularly, the invention relates to modulation of such immune stimulation by modification of oligonucleotides.

2. Summary of the Related Art

Bacterial DNA and synthetic oligodeoxynucleotides containing unmethylated CpG-dinucleotide motifs (CpG oligos) activate the immune system as manifested by proliferation of B cells, activation of antigen-presenting cells (macrophages and dendritic cells), and the secretion of IL-6, IL-12, TNF-□. and IFN-□.¹⁻³ The presence of a CpG-motif can cause the immunostimulatory activity and the position of the CpG-motif and the sequences flanking the CpG-motif play a critical role in determining the immunostimulatory activity of CpG oligos.¹⁻⁴ It has recently been shown that unmethylated CpG-motifs of DNA and oligonucleotides are recognized by a transmembrane protein, toll-like receptor 9 (TLR9), which ultimately leads to the stimulation of stress kinase pathways, including activation of NF-□B and induction of various cytokines.⁵ Alternately, Chu and coworkers have shown that the CpG-DNA triggers DNA protein kinase activation, which phosphorylates IkB-kinaseB, leading to the activation of NF-□B, which further leads to the production of proinflammatory cytokines.⁶ It is not clear whether the two pathways are activated sequentially or in parallel leading to a common function of activating the NF-□B pathway. Nonetheless, the use of CpG oligos as antitumor, antiviral, antibacterial, and anti-inflammatory agents and as adjuvants in immunotherapy has been reported.⁷⁻¹³

Our laboratory has been studying the effects of sequence and structural changes in the flanking sequences that potentiate or suppress immunostimulatory activities of CpG oligos. We have shown that replacement of deoxynucleosides in a CpG-motif with 2′-O-methylribonucleosides suppresses immunostimulatory activity.¹⁴ In contrast, the substitution of one or two 2′-deoxynucleosides adjacent to the CpG-motif with 2′- or 3′-O-methylribonucleosides on the 5′-side causes a decrease in immunostimulatory activity, while the same substitutions have an insignificant effect when they were placed on the 3′-side of the CpG-motif.¹⁵ However, the substitution of a deoxynucleoside two or three nucleosides away from the CpG-motif on the 5′-side with one or two 2′-O-methoxyethoxy- or 2′- or 3′-O-methylribonucleosides results in a significant increase in immunostimulatory activity.¹⁶ In addition, we have also demonstrated that an accessible 5′-end, but not 3′-end, is critical for immunostimulatory activity of CpG oligos.¹⁷

Our earlier studies showed that substitution of a methyl group for an unbridged oxygen on the phosphate group between the C and G of a CpG-motif (FIG. 1) suppresses immunostimulatory activity, suggesting that a negative charge on the phosphate group is essential for receptor recognition, interaction, and subsequent immunostimulatory activity.¹⁴ In our continuing pursuit to understand the molecular and structural determinants of immunostimulatory activity of CpG oligos, in the present study we examined the effect of internucleoside charge neutralization by substituting a negatively charged phosphorothioate linkage with a non-ionic methylphosphonate linkage (FIG. 1) in both the 5′- and the 3′-flanking sequences of the CpG-motif.

### BRIEF SUMMARY OF THE INVENTION

Although it is well established that CpG-motifs within certain specific sequence contexts elicit immunostimulatory activity,¹⁻⁴ there are no reports delineating the molecular and structural requirements that determine the immunostimulatory functions of CpG oligos. As the synthetic CpG oligos are rapidly advancing to human clinical trials for various disease indications and as vaccine adjuvants,²¹ it is important to understand the molecular and structural determinants of immunostimulatory activity of CpG oligos in order to develop potent CpG oligo mimics.²² This is the first report in which the role of internucleoside negative charge in the flanking sequences to the CpG-motif is studied by substituting with a non-ionic linkage to improve the immunostimulatory potential of CpG oligos.

Substitution of a methyl group for an unbridged oxygen on the phosphate group between the C and G of a CpG-motif (FIG. 1) suppresses immunostimulatory activity of a CpG oligo, suggesting that negative charge on phosphate group is essential for receptor recognition, interaction, and subsequent immunostimulatory activity.¹⁴ The present study suggests that non-ionic phosphate linkages in the flanking sequences may enhance, suppress or maintain the immunostimulatory activity, compared with an unmodified CpG oligo, depending on the position of the substitution. In general, substitution at the first three internucleoside linkages adjacent to the CpG-motif on the 5′-side suppressed mouse spleen cell proliferation, splenomegaly, and secretion of IL-12 and IL-6, suggesting negative charge at these internucleoside linkages is important for recognition and interaction of CpG oligos with the receptor in the immunostimulatory signaling pathway.

In contrast, substitution with non-ionic linkages at the fifth and/or sixth internucleoside linkages adjacent to the CpG-motif on the 5′-side significantly enhanced mouse spleen cell proliferation, splenomegaly, and IL-12 and IL-6 production compared with parent CpG oligo. This result suggests that the presence of non-ionic internucleoside linkages at these positions permits tighter interaction between the receptor and modified CpG oligo, leading to increased immunostimulatory activity. These results are also in support of our earlier studies^{15,16} in which a substitution of one or two deoxynucleosides with lipophilic 2′- or 3′-O-methylribonucleosides on the 5′-side of the CpG-motif increased immunostimulatory activity. Taken together these results suggest that introduction of more lipophilic structural changes introduced about three to five nucleosides away from the CpG-motif in the 5′-flanking sequence facilitates stronger interaction of the CpG oligo with the receptor, leading to higher immunostimulatory activity.

The introduction of non-ionic internucleoside linkage in the 3′-flanking sequence to the CpG-motif did not show a significant difference in the splenomegaly compared with parent oligo, though spleen cell proliferation was considerably affected depending on the position of non-ionic linkage. Unlike in the case of 5′-substitutions, it is difficult to correlate IL-12 and IL-6 secretion patterns with cell proliferation and splenomegaly. For example, oligos 8, and 11-13 had much lower affect on spleen cell proliferation but induced higher levels of IL-12 secretion than did the parent oligo, suggesting the modifications in the 3′-flanking sequences might alter recognition of the receptor leading to different cytokine production profiles.

Increased immunostimulatory activity of non-ionic methylphosphonate linkage containing CpG oligos could also be the result of increased internalization or nuclease resistance compared with unmodified parent oligos. However, in such cases all the oligos that contained one methylphosphonate linkage should have shown similar level of cell proliferation, splenomegaly, and cytokine induction and the results presented here do not support this. In addition, the extent of immunostimulatory activity is not dependent on the number of methylphosphonate linkages (compare one linkage vs two linkages) but dependent on the position of substitution. Therefore, the results strongly support that the modifications incorporated influence the recognition and/or interaction of the CpG oligos with the receptor but not internalization or their stability against nucleases.

In conclusion, the immunostimulatory activity of a CpG oligo can be modulated by site-specific incorporation of a non-ionic methylphosphonate linkage in the flanking sequences of the CpG-motif. In addition, combining these modifications with recently reported unnatural YpG- and CpR-motif containing oligos²³ may lead to the development of potent immunomodulatory agents with greater bioavailability than the unmodified natural CpG-motif containing oligos. Several such oligos developed through combinatorial approaches are currently under study.

### BRIEF DESCRIPTION OF THE DRAWINGS

FIG. 1. Chemical structure of a dinucleotide showing internucleoside linkage. B stands for nucleobase A, C, G or T. R is O⁻ in natural phosphodiester linkage, S⁻ in phosphorothioate linkage or CH₃ in a methylphosphonate linkage. Note phosphodiester and phosphorothioate linkages are anionic and methylphosphonate linkage is non-ionic.

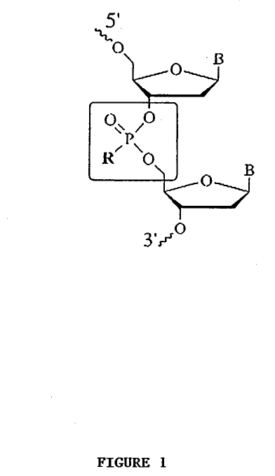

FIG. 2. Immunostimulatory activity of CpG oligos 1-7. A. BALB/c mouse spleen cell proliferation in cell cultures at 0.1 □g/mL concentration, B. splenomegaly (enlargement of spleen) in BALB/c mice at 5 mg/kg dose of CpG oligo, C. IL-12, and D. IL-6 secretion in BALB/c mouse spleen cultures at 0.1 □g/mL concentration of CpG oligos.

FIG. 3. Immunostimulatory activity of CpG oligos 1, and 8-13. A. BALB/c mouse spleen cell proliferation in cell cultures at 0.1 □g/mL concentration, B. splenomegaly in BALB/c mice at 5 mg/kg dose of CpG oligo, C. IL-12, and D. IL-6 secretion in BALB/c mouse spleen cultures at 0.1 □g/mL concentration of CpG oligos.

### DETAILED DESCRIPTION OF THE PREFERRED EMBODIMENTS

We have synthesized oligonucleotides containing one or two methylphosphonate linkages in either the 5′- or the 3′-flanking sequence of the CpG-motif (Table 1) and studied the effect of these agents on immunostimulatory activity. Oligo 1 is a phosphorothioate oligodeoxynucleotide that contained a CpG motif in a hexameric sequence context, GACGTT, that is recognized by mouse immune system and induces cell proliferation, cytokine production in mouse cell cultures, and splenomegaly in mice.⁴ Oligos 2-6 contained a methylphosphonate linkage in place of a phosphorothioate linkage in the 5′-flanking sequence of the CpG-motif at indicated positions (Table 1). Oligo 7 contained two methylphosphonate linkages in the 5′-flanking sequence (Table 1). Similarly, oligos 8-12 contained one methylphosphonate linkage and oligo 13 contained two methylphosphonate linkages in the 3′-flanking sequence. We have designated these positions as 1 to n with 1 being the closest to the C or G of the CpG-motif towards the 5′- or 3′-side with a 5′- or 3′-designation, respectively. For example, the description of a methylphosphonate linkage at 5′-1-internucleoside position corresponds to oligo 2, which had a non-ionic methylphosphonate linkage adjacent to the C-nucleoside on the 5′-side of the CpG-motif. Similarly, substitution at the 3′-2-internucleoside position refers to oligo 8, in which a methylphosphonate linkage was placed on the 3′-side of the CpG-motif (Table 1). Double substitutions in oligos 7 and 13 are described as 5′-5,6- and 3′-6,7-internucleoside positions, respectively, for discussion purposes.

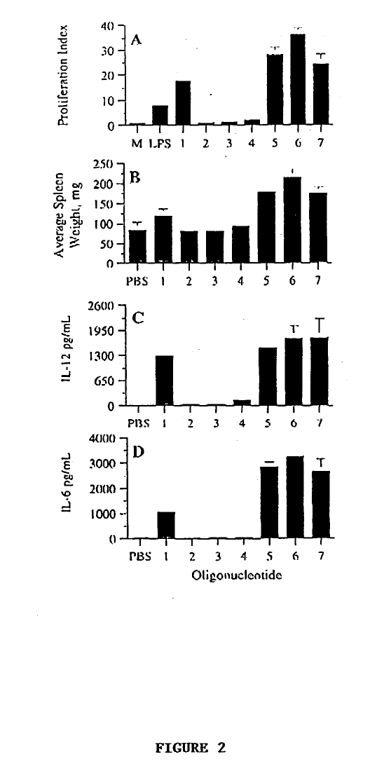

All the oligos were examined for their ability to induce cell proliferation in BALB/c mouse spleen cell cultures. All the oligos showed a concentration-dependent lymphocyte proliferation. The parent oligo which had no non-ionic linkage, showed a proliferation index of 17.6±1.1 at a concentration of 0.1 □g/mL (FIG. 2A). Replacing a negatively charged phosphorothioate internucleoside linkage at 5′-1-, 2-, or 3-position with a non-ionic methylphosphonate linkage (oligos 2-4, respectively) resulted in the loss of lymphocyte proliferatory activity (FIG. 2A). Substitution of a nonionic linkage at 5′-4- or 5-internucleoside position (oligos 5 and 6, respectively) showed proliferation indices of 28.1±3.1 and 36.0±2.8, respectively, at 0.1 □g/mL concentration (FIG. 2A). These proliferation index values for oligos 5 and 6 are about 60% and 104% higher, respectively, compared with proliferation index of parent oligo 1. Oligo 7, which had two methylphosphonate linkages at 5′-5,6-internucleoside positions also showed an increased proliferatory index of 24.3±3.9 at 0.1 □g/mL concentration (FIG. 2A), which was about 38% higher compared with parent oligo 1.

Oligos 1-7 were injected to BALB/c mice at a dose of 5 mg/kg and examined for increase in spleen weight as a result of oligo treatment as described in Experimental Procedures Section. The increase in spleen weight compared with control group injected with PBS was considered to be the result of immunostimulatory activity of CpG oligos as described earlier.²⁰ The results obtained from these mice experiments (FIG. 2B) are complementary to those obtained from cell culture experiments (FIG. 2A). Oligo 1, which did not have a non-ionic linkage showed about 44% increase in spleen weight compared with control mice treated with PBS (FIG. 2B). Oligos 2-4 at the same dose showed no/insignificant change in spleen enlargement compared with the control group of mice (FIG. 2B). Oligos 5-7 at the same dose showed a higher spleen enlargement of about 48%, 76%, and 45%, respectively, compared with parent oligo 1 (FIG. 2B).

We then tested the CpG oligos for their ability to induce IL-12 and IL-6 production in BALB/c mouse spleen cell cultures. Oligos 1, and 5-13 induced IL-12 and IL-6 in a dose-dependent manner (Table 1). Additionally, cytokine induction is also dependent on the position of the non-ionic linkage present in the flanking sequence. The parent oligo 1, which does not have any modification, produced 1292 and 1067 pg/mL of IL-12 and IL-6, respectively, at a concentration of 0.1 □g/mL. Oligos 2-4, which have a non-ionic linkage at the 5′-1-, -2, or -3-internucleoside position, respectively, showed similar IL-12 and IL-6 levels as that of media (PBS) control. The replacement of negative charge with a non-ionic linkage at the 5′-4- or -5-internucleoside position (oligos 5 and 6) resulted in a substantial increase in both IL-12 and IL-6 production compared with parent oligo 1 at the same concentration (FIGS. 2C and 2D). Similarly, an increased level of IL-12 and IL-6 production was also noted with oligo 7, which had two non-ionic linkages at 5′-4 and 5-internucleoside positions, compared with parent oligo 1.

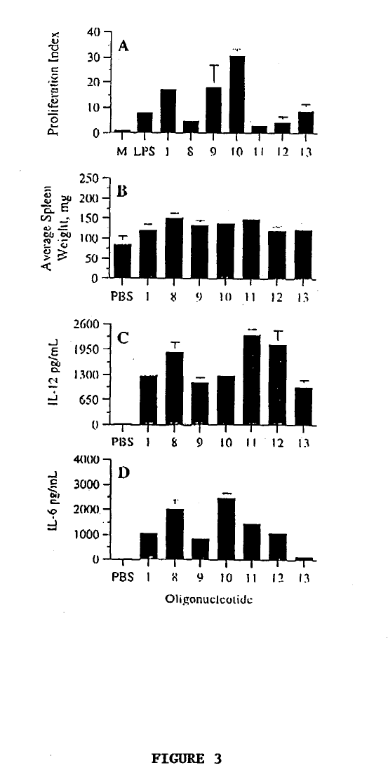

Similar to 5′-flanking region substitutions, we have also incorporated non-ionic methylphosphonate substitutions in the 3′-flanking sequence of the CpG-motif (oligos 8-13, Table 1) and studied for immunostimulatory activity in lymphocyte cultures and splenomegaly in mice. FIG. 3A shows proliferation indices observed for oligos 1 and 8-13 at a concentration of 0.1 □g/mL in BALB/c splenocyte cultures. Oligos 8, 11, and 12, which had a methylphosphonate internucleoside linkage at positions 3′-2-, -5-, and -6-internucleoside positions, showed a lower cell proliferation index compared with parent oligo 1. Oligo 9, which had a methylphosphonate linkage at the 3′-3-internucleoside position, showed similar proliferation index of 18.2±8.9 comparable with that of parent oligo 1. Oligo 10, which had a methylphosphonate linkage at the 3′-4-internucleoside position, showed a proliferation index of 30.9±2.5, which is significantly higher than that observed for parent oligo 1. Oligo 13 with two methylphosphonate linkages at 3′-6,7-internucleoside positions showed a proliferation index of 9.0±2.5, which is about 50% lower proliferation index than that observed with parent oligo 1.

The ability of oligos 8-13 to induce splenomegaly in BALB/c mice was examined at a dose of 5 mg/kg. The results are shown in FIG. 3B. These results suggest that incorporation of a non-ionic methylphosphonate linkage in the 3′-flanking sequence to the CpG-motif has caused equal or slightly increased spleen enlargement compared with parent oligo 1.

We also tested the ability of oligos 8-13 to induce IL-12 and IL-6 production in mouse spleen cell cultures. As in the case of 5′-modified oligos, 3′-modified oligos also induced both IL-12 and IL-6 production, which was dependent on the concentration of the oligo and position of the modification in the oligo (Table 1). Oligo 8, which had a non-ionic linkage at 3′-1-internucleoside position, showed high levels of IL-12 and IL-6 production (FIGS. 3C and D), although it induced lower cell proliferation compared with the parent oligo (FIG. 3A) at the same concentration. In general, slightly lower levels of IL-12 and IL-6 secretion was observed with oligo 9, which had a non-ionic linkage at 3′-2-internucleoside position, although it showed cell proliferation and splenomegaly in mice equal to that produced by the parent oligo 1. Oligo 10, which had a non-ionic linkage at the 3′-3-internucleoside position, produced similar levels of IL-12 and significantly elevated levels of IL-6 compared with the parent oligo 1. Oligos 11 and 12, which had a non-ionic linkage, each induced higher levels of IL-12 and IL-6 than did oligo 1. Oligo 13, which had two non-ionic linkages, produced lower levels at lower concentrations and slightly higher levels of IL-12 at higher concentrations than did oligo 1. However, it produced substantially lower levels of IL-6 than did parent oligo 1.

[Replace with Table 1]

The following examples are intended to further illustrate certain preferred embodiments of the invention and are not intended to limit the scope of the invention.

### EXAMPLE 1

Oligodeoxynucleotide Synthesis and Purification

Oligonucleotides were synthesized using □-cyanoethylphosphoramidite chemistry on a PerSeptive Biosystem's 8900 Expedite DNA synthesizer on 1 □mole scale. Phosphoramidites of dA, dG, dC and T were obtained from PerSeptive Biosystems. The required methylphosphonamidites were purchased from Glen Research. Beaucage reagent was used as an oxidant to obtain phosphorothioate backbone modification. At the required position, methylphosphonamidite monomer was incorporated and oxidized with iodine/H₂O/THF/lutidine reagent as reported earlier.¹⁸ After the synthesis, oligos were deprotected as required, purified by HPLC, converted to sodium form and dialyzed against distilled water. Then the oligos were lyophilized and redissolved in distilled water and the concentrations were determined by measuring the UV absorbance at 260 nm. PS-oligos were characterized by CGE and MALDI-TOF mass spectrometry (Brucker Proflex III MALDI-TOF mass spectrometer with 337 nm N2 laser) for purity and molecular mass, respectively.

### EXAMPLE 2

Mouse Lymphocyte Proliferation Assay

Lymphocytes obtained from BALB/c mouse (4-8 weeks) spleens were cultured in RPMI complete medium as described earlier.^{14,19} The cells were plated in 96-well dishes at a density of 10⁶ cells/mL in a final volume of 100 □L. The CpG oligos or LPS (lipopolysaccharide, a positive control) were added to the cell culture in 10 □L of TE buffer (10 mM Tris-HCl, pH 7.5, 1 mM EDTA) at a final concentration of 0.1, 0.3, 1.0 and 3.0 □g/mL. The cells were then incubated at 37° C. After 44 h, 1 □Ci ³H-uridine (Amersham) was added to the culture in 20 □L of RPMI medium, and the cells were pulse-labeled for another 4 h. The cells were harvested by automatic cell harvester and the filters were counted by a scintillation counter. The experiments were performed two or three times for each oligo in triplicate at each concentration. The averages were calculated, normalized and presented as proliferation index.

### EXAMPLE 3

Assays for IL-12 and IL-6 Secretion in Mouse Spleen Cell Cultures

The secretion of IL-12 and IL-6 in BALB/c mouse spleen cell cultures was measured by sandwich ELISA. The required reagents including cytokine antibodies and cytokine standards were purchased form PharMingen. ELISA plates (Costar) were incubated with appropriate antibodies at 5 □g/mL in PBSN buffer (PBS/0.05% sodium azide, pH 9.6) overnight at 4° C. and then blocked with PBS/10% FBS at 37° C. for 30 min. Cell culture supernatants and cytokine standards were appropriately diluted with PBS/10% FBS, added to the plates in triplicate, and incubated at 25° C. for 2 hr. Plates were overlaid with 1 □g/mL appropriate biotinylated antibody and incubated at 25° C. for 1.5 hr. Then the plates were washed extensively with PBS/0.05% Tween 20 and then further incubated at 25° C. for 1.5 hr after adding streptavidine conjugated peroxidase (Sigma). Then the plates were developed with chromatin (Kirkegaard and Perry) and the color change was measured on a Ceres 900 HDI Spectrophotometer (Bio-Tek Instruments). The levels of IL-12 and IL-6 in the cell culture supernatants were calculated from the standard curve constructed under the same experimental conditions for IL-12 and IL-6.

### EXAMPLE 4

Mouse Splenomegaly Assay of CpG Oligos

Female BALB/c mice (4-6 weeks, 19-21 gm) were divided in to different groups with four mice in each group. Oligonucleotides were dissolved in sterile PBS and administered intraperitoneally to mice at a dose of 5 mg/kg. After 72 hr, mice were sacrificed and spleens were harvested and weighed.

1. A method for enhancing the immunostimulatory effect of a CpG-containing immunostimulatory oligonucleotide, the method comprising substituting a non-ionic internucleoside linkage at a position five to six nucleosides 5′ to the CpG.

2. A method for reducing the immunostimulatory effect of a CpG-containing immunostimulatory oligonucleotide, the method comprising substituting a non-ionic internucleoside linkage at a position one to three to nucleosides 5′ to the CpG.
